(19) United States
(12) Patent Application Publication
(10) Pub. No.: US 2020/0041418 A1
(43) Pub. Date: Feb. 6, 2020

(54) NANOPLASMONIC PAPER SUBSTRATE FOR IDENTIFICATION OF FENTANYL AND FENTANYL-RELATED COMPOUNDS

(71) Applicant: The Texas A&M University System, College Station, TX (US)

(72) Inventors: Hung-Jen Wu, College Station, TX (US); Joshua D. Weatherston, College Station, TX (US)

(21) Appl. No.: 16/525,135

(22) Filed: Jul. 29, 2019

Related U.S. Application Data
(60) Provisional application No. 62/712,696, filed on Jul. 31, 2018.

(51) Int. Cl. G01N 21/65; G01J 3/44; D21H 19/02

(52) U.S. Cl. CPC G01N 21/658; C07D 211/58; D21H 19/02; G01J 3/44

(57) ABSTRACT

In an embodiment, a method to detect analytes, the method including contacting a sample on a substrate, where the substrate is a nanoplasmonic paper, performing surface-enhanced Raman scattering detection with paper chromatography separation on the substrate, and identifying at least one analyte in the sample. In a further embodiment, an apparatus to detect analytes, the apparatus including a vacuum pump coupled to a filtration unit operable to collect solid particles off an object and the filtration unit including a filter substrate, where the filter substrate includes nanoplasmonic paper.

### CROSS-REFERENCE TO RELATED APPLICATIONS

This patent application claims priority from, and incorporates by reference the entire disclosure of, U.S. Provisional Patent Application No. 62/712,696 filed on Jul. 31, 2018.

### BACKGROUND

This section provides background information to facilitate a better understanding of the various aspects of the disclosure. It should be understood that the statements in this section of this document are to be read in this light, and not as admissions of prior art.

Fentanyl is a dangerous, powerful, Schedule II narcotic responsible for an epidemic of overdose deaths within the United States. Fentanyl is up to 50 times more potent than heroin. It is extremely dangerous to anyone who may come into contact with it. Although fentanyl, a synthetic opiate painkiller, is legitimately used by doctors to treat patients with severe pain, the synthetic drug is now flooding the streets as a more potent and deadly alternative to heroin and prescription painkillers. Fentanyl is often mixed with heroin to increase potency, but dealers and buyers may not know exactly what they are selling or ingesting. Many users underestimate the potency of fentanyl, significantly raising the risk of overdose.

Fentanyl is not only dangerous for the drug users, but for law enforcement, public health workers, first responders, and parcel-handling personnel who unknowingly come into contact with it in its different forms. Fentanyl can be absorbed through the skin or accidental inhalation of airborne powder. In particular, the Drug Enforcement Agency (DEA) is concerned about law enforcement coming into contact with fentanyl on the streets during the course of narcotics work. Canine units are particularly at risk of immediate death from inhaling fentanyl during their work. Just 2 mg of fentanyl can cause death in most people, but this varies greatly. Fentanyl and its analogs come in several forms including, but not limited to, powders, blotter papers, tablets, and sprays.

The current outbreak involves not just fentanyl, but also fentanyl compounds. The outbreak encompasses virtually the entire United States, resulting in thousands of deaths and involves a wide array of individuals, including new and experienced abusers. In the last three years, the DEA has seen a significant resurgence in fentanyl-related seizures. In addition, the DEA has identified at least 15 other deadly, fentanyl-related compounds. Some fentanyl cases have been significant, particularly in the Northeast and in California. In a previous seizure resulting from a routine traffic stop, 40 kilograms of fentanyl, which was initially believed to be bricks of cocaine, wrapped into blocks hidden in buckets and immersed in a thick fluid was confiscated. Recent seizures of counterfeit hydrocodone or oxycodone tablets have also revealed the presence of fentanyl. These fentanyl tablets are marked to mimic the authentic narcotic prescription medications and have led to multiple overdoses and deaths.

Due to the increasing use of fentanyl and fentanyl-related compounds, and considering its extreme toxicity, there exists a need for law enforcement, first responders, and parcel packaging handlers, among others, to have a reliable method for detection of fentanyl and fentanyl-related compounds, even when only present in trace amounts. The present disclosure seeks to address this need by providing reliable substrates, and methods of use thereof, for testing for, and detecting, chemical substances and, additionally, for devices for use of these substrates.

### SUMMARY OF THE INVENTION

This summary is provided to introduce a selection of concepts that are further described below in the Detailed Description. This summary is not intended to identify key or essential features of the claimed subject matter, nor is it to be used as an aid in limiting the scope of the claimed subject matter.

In an embodiment, a method to detect analytes, the method including contacting a sample on a substrate, where the substrate is a nanoplasmonic paper, performing surface-enhanced Raman scattering detection with paper chromatography separation on the substrate, and identifying at least one analyte in the sample. In some embodiments, the nanoplasmonic paper includes glass microfiber filter paper coated with silver nanoparticles. In some embodiments, the silver nanoparticles are coated on the nanoplasmonic paper via a silver mirror reaction. In some embodiments, the substrate includes a compound to enhance affinity between the substrate and the at least one analyte to thereby increase detection limit. In some embodiments, the compound can include, without limitation, organic compounds, inorganic compounds, thiols, functional groups, functional molecules, 1-butanethiol, or combinations thereof. In some embodiments, the sample is a mixed-analyte sample including a plurality of analytes. In some embodiments, the contacting includes collecting solid particles of the sample onto the substrate via vacuum. In some embodiments, the nanoplasmonic paper is extended between a first end and a second end of a filter cartridge adaptable to be coupled to a vacuum, and where a solvent reservoir is coupled to the first end. In some embodiments, the performing includes eluting a solvent through the nanoplasmonic paper, separating components of the sample, and identifying the components of the sample via spectroscopy. In some embodiments, the at least one analyte can include, without limitation, natural opioids, synthetic opioids, opioid residues, fentanyl, fentanyl-related compounds, carfentanil, acetyl fentanyl, cannabinoids, synthetic cannabinoids, or combinations thereof.

In a further embodiment, an apparatus to detect analytes, the apparatus including a vacuum pump coupled to a filtration unit operable to collect solid particles off an object and the filtration unit including a filter substrate, where the filter substrate includes nanoplasmonic paper. In some embodiments, the filter substrate is disposed with a filter cartridge, the filter cartridge including a first end and a second end, a solvent reservoir coupled to the first end, and the nanoplasmonic paper extending on a surface of the filter cartridge from the solvent reservoir to the second end. In some embodiments, the solvent reservoir is operable to be punctured to elute a solvent through the nanoplasmonic paper thereby separating components of the solid particles. In some embodiments, the filter cartridge is operable to be inserted into a handheld Raman spectrometer. In some embodiments, the apparatus includes a sorting surface and at least one air jet in fluid communication with the object and the filtration unit. In some embodiments, the object can include, without limitation, a surface, luggage, a mail parcel, carpet, wood, cloth, or combinations thereof. In some embodiments, the filter substrate includes a compound to enhance affinity between the filter substrate and analytes in the solid particles to thereby increase detection limit of the analytes. In some embodiments, the compound can include, without limitation, organic compounds, inorganic compounds, thiols, functional groups, functional molecules, 1-butanethiol, or combinations thereof. In some embodiments, the nanoplasmonic paper includes glass microfiber filter paper coated with silver nanoparticles. In some embodiments, the silver nanoparticles are coated on the nanoplasmonic paper via a silver mirror reaction.

### BRIEF DESCRIPTION OF THE DRAWINGS

A more complete understanding of the subject matter of the present disclosure may be obtained by reference to the following Detailed Description when taken in conjunction with the accompanying Drawings wherein:

FIG. 1A and FIG. 1B illustrate nanopaper characterization results: size, FIG. 1A, and spacing, FIG. 1B, distributions of silver nanoparticles on nanopaper, based on SEM micrographs. 100 distinguishable particles were chosen and their diameters were measured, along with the distance to the nearest distinguishable particle.

FIG. 2 illustrates Raman signal comparison of 10⁻⁴ M R6G adsorbed on nanopaper (solid line) and 4×10⁻⁴ M R6G adsorbed on glass microfiber filter (dashed line, scaled by 1000 times for visibility).

FIG. 3 illustrates molecular structure of 4 organic dyes at pH ˜2.5.

FIG. 4A and FIG. 4B illustrates results of PC-SERS on a four-dye mixture. FIG. 4A shows an intensity profile of the characteristic peak for each dye as a function of R_{f} (lower axis) and migration distance (upper axis). Each profile is normalized by its own minimum and maximum values to clearly show species localization. FIG. 4B shows reference SERS spectra of the four dyes (scaled and shifted for clarity). Peaks used to construct species profiles are denoted *.

FIG. 5 illustrates spectral comparison of solid phase (long-dash and solid lines) and SERS (short-dashed and dotted lines) spectra for reference lycopene and β-carotene. Molecular structures are shown for each. Spectra are scaled and offset for clarity.

FIG. 6 illustrates SERS spectra of β-carotene (a) and lycopene (b) on nanopaper, compared to PC-SERS spectra of tomato (c) and (d), and carrot (e) and (f). For clear comparison, (a)-(e) are offset and scaled with reference to the major carotenoid peak near 1154 cm⁻¹. Because (f) showed no carotenoid signal, it was instead scaled to match the background signal of (e). Vertical guides are placed where (a) and (b) exhibit the largest variation.

FIG. 7 illustrates PC-SERS of juice extracts containing carotenoids, with lycopene and β-carotene references. Separation was performed on nanopaper, using absolute ethanol as the mobile phase. Intensities are relative to the nanopaper background signal at 900 cm⁻¹, and each profile is normalized from 0 to 1 for clarity. Total chromatogram length varies between samples; each bar represents 1 mm distance.

FIG. 8 illustrates additional PC-SERS results for carotenoid reference experiments. Three additional replicates were performed for lycopene and β-carotene references, to demonstrate repeatability. Samples were prepared and analyzed in the same manner as those in FIG. 7. Linear spatial resolution: 250 μm.

FIG. 9 illustrates evidence of lycopene contamination by other carotenoid. SERS spectra of β-carotene (a) and lycopene (b) references spotted on nanopaper; PC-SERS spectra from R_{f}=0.95 (c) and R_{f}=0.05 (d) of reference lycopene developed in ethanol. The peak shifts between (c) and (d) resemble the shifts between (a) and (b), particularly in the 1500-1550 cm⁻¹ region.

FIG. 10 shows an illustration of nanopaper substrate PC-SERS according to an embodiment of the present disclosure.

FIG. 11A, FIG. 11B and FIG. 11C illustrate an overview of a VF-SERS system. FIG. 11A shows nanopaper synthesis, with SEM images of nanopaper morphology (scale bars=3 μm). FIG. 11B shows nanopaper is used as a filter to collect particulates from the air. FIG. 11C shows simple and rapid on-filter separation enables identification of filtered chemical species.

FIG. 12 illustrates a schematic of a vacuum collection chamber according to an embodiment of the present disclosure.

FIG. 13A, FIG. 13B, FIG. 13C and FIG. 13D illustrates Raman spectra of powders used in VF-SERS demonstrations. FIG. 13A shows SERS spectrum of pure R6G captured on nanopaper by vacuum filtration. FIG. 13B shows solid spectrum of topsoil deposited on nanopaper via vacuum filtration. FIG. 13C shows solid spectrum of lactose deposited on nanopaper via vacuum filtration. FIG. 13D shows R6G deposited on nanopaper via vacuum filtration before (dash-dot line) and after (solid line) solvent elution.

FIG. 14 illustrates particle size distribution of the 3 powder species used in VF-PC-SERS experiments (the topsoil distribution excludes some particles that were beyond the instrumental range).

FIG. 15 illustrates R6G detection by VF-PC-SERS. Each point represents the mean intensity at 612 cm⁻¹ for a nanopaper sample strip. The detection threshold is set at 3 standard deviations above the mean of the negative controls.

FIG. 16 illustrates SERS intensity vs mass of R6G deposited on nanopaper. Error bars represent the standard error of spectral intensities at each level (n=5).

FIG. 17 illustrates sensitivity of R6G detection by VF-PC-SERS with 20 s vacuum filtration duration. 10 trials were performed for each loading.

FIG. 18 illustrates specificity analysis of VF-PC-SERS. Each point shows the mean SERS intensity at 612 cm⁻¹ for a nanopaper sample strip. Each experiment was performed in a chamber loaded with 5 mg R6G mixed with the diluent loading displayed. The limit of blank is carried over from FIG. 15.

FIG. 19A, FIG. 19B and FIG. 19C illustrate Raman spectrum for a sample placed inside a vacuum chamber with the vacuum line blocked by a nanopaper filter. FIG. 19A illustrates Raman spectrum of the clean nanopaper. FIG. 19B illustrates Raman spectrum of the nanopaper after filtering the garden soil. FIG. 19C illustrates the Raman spectrum of the nanopaper after filtering the mixture of R6G and garden soil. After applying a drop of solvent, the background signal could be removed, and the clear R6G Raman spectrum was observed.

FIG. 20A and FIG. 20B illustrates SERS results of vacuum collection for R6G (FIG. 20A: Full results, FIG. 20B: detail of low concentration region). Dashed line represents the detection threshold μb+3σ_{b}.

FIG. 21 illustrates SERS results for trace fentanyl detection. Spectra are shown for fentanyl adsorbed on nanopaper modified with 1-butanethiol (a), a control nanopaper modified with 1-butanethiol (b), and the difference spectrum (a)-(b). Solid fentanyl spectrum is given for comparison (s). Spectra have been offset for visual clarity.

FIG. 22A, FIG. 22B, FIG. 22C and FIG. 22D illustrate a disposable threat detection cartridge. FIG. 22A shows a plastic cartridge containing an organic solvent reservoir and nanopaper. The nanopaper is a glass fiber paper decorated with silver nanoparticles. This nanopaper has multiple functions. First, it serves as a vacuum filter paper for sample collection. Second, it is the stationary phase for paper chromatography. Third, the nanoparticles on the nanopaper enhance Raman signals of chemicals, improving detection sensitivity. FIG. 22B shows SEM image of nanopaper (scale bar=4 μm). FIG. 22C shows the cartridge can be installed in commercial vacuum cleaners, including handheld or robotic vacuum cleaners. Dusts and chemicals (e.g., drug, explosive, etc.) on the floor or in the air will be collected on the nanopaper. FIG. 22D shows separating dust and chemicals to improve the detection limit. The solvent reservoir can be punctured to release organic solvents (e.g., methanol or ethanol). The components move with the flowing solvent, leading to separations of components. The cartridge can then be inserted into the commercial handheld Raman spectrometer to identify the chemicals via their fingerprint spectra.

FIG. 23 illustrates a detection system according to an embodiment of the present disclosure to detect contaminated parcels before unloading (e.g. during sorting).

FIG. 24 illustrates a detection system according to an embodiment of the present disclosure that inspects parcels one-by-one on a sorting railroad.

### DETAILED DESCRIPTION

It is to be understood that the following disclosure provides many different embodiments, or examples, for implementing different features of various embodiments. Specific examples of components and arrangements are described below to simplify the disclosure. These are, of course, merely examples and are not intended to be limiting. The section headings used herein are for organizational purposes and are not to be construed as limiting the subject matter described.

Surface-enhanced Raman scattering (SERS) is a powerful analytical tool which enables the detection and identification of analytes adsorbed on nanostructured noble metals. However, SERS analysis of complex mixtures can be challenging due to spectral overlap and interference. The present disclosure seeks to demonstrate a method to assist the identification of mixed-analyte samples by coupling SERS detection with paper chromatography (PC) separation on a nanoplasmonic paper substrate. The physical separation of a complex mixture via PC allows the mixture's complex SERS spectrum to be replaced with a collection of simple spectra. This in turn simplifies the identification of analytes by eliminating sources of spectral interference. The methods presented herein are relatively fast, simple, and inexpensive. When performed correctly it results in increased confidence in identification of complex mixtures compared to PC or SERS alone. The present disclosure seeks to leverage the nanoplasmonic paper substrates and methods of use thereof in order to accurately identify compounds, such as fentanyl.

In some embodiments, the present disclosure utilizes a paper-like substrate that simultaneously functions as a PC stationary phase support and as a SERS enhancing substrate. This nanoplasmonic paper (“nanopaper”) substrate can be produced by a facile one-step synthesis which forms a dense film of silver nanoparticles on glass microfiber filter paper. In some embodiments, the nanoparticles are metal nanoparticles. In some embodiments, the present disclosure seeks to illustrate a nanopaper substrate, which can be a glass microfiber filter paper that has been coated with a dense layer of silver nanoparticles via, for example, the silver mirror reaction, and demonstrate methods of use of nanopaper substrate in coupled paper chromatography-SERS (PC-SERS) identification, to identify compounds, such as, for example, fentanyl, or fentanyl-related compounds (e.g., carfentanil and acetyl fentanyl), natural opioids, or synthetic derivatives thereof.

In various embodiments, the methods disclosed herein can use silver-coated glass microfiber filter paper, referred to herein as “nanopaper”, as a combination paper chromatography/SERS substrate that can further include additional functional molecules, such as, but not limited to, 1-butanethiol, to enhance the detection of trace amounts of fentanyl as well as improve the separation efficiency of paper chromatography.

As PC-SERS has not seen wide adoption as an analytical method partially because previously reported PC-SERS substrates required multiple complicated synthetic steps to produce, limiting accessibility, in some embodiments of the present disclosure, nanopaper presented herein has a facile synthesis, and it is a portable, durable, and effective PC-SERS substrate that can be utilized in chemical detection methods.

In some embodiments, nanopaper can be easily produced on a large scale with considerations such as packaging and handling taken into account. The present disclosure seeks to identify PC-SERS as a versatile method which can be used to develop screening assays in a number of fields, including medicine, biology, forensic science, environmental science, food and drug testing, and others. In a particular embodiment, the PC-SERS methods disclosed herein can be utilized for identification of fentanyl. Furthermore, researchers could use the nanopaper presented herein to develop PC-SERS screening assays simply by identifying the proper PC mobile phase for an analyte of interest. Once an assay is developed, nanopaper could be packaged as “test strips” and bundled with the appropriate mobile phase as an assay kit, and can include, for example, “test strips” and mobile kits for the identification of fentanyl, or fentanyl-related compounds (e.g., carfentanil and acetyl fentanyl), natural opioids, synthetic derivatives thereof, synthetic opioids, opioid residues, or combinations of the same and like.

In various embodiments, the methods presented herein have application in many fields, and currently used technologies vary depending on the specific application. Generally speaking, the current technologies capable of high-confidence identification of mixed-analyte samples include coupled separation-detection methods. Predominant separation procedures involve liquid/high-performance liquid chromatography (LC/HPLC), gas chromatography (GC), thin layer chromatography (TLC), and the like. Other common protocols include electrophoresis and immunoprecipitation. Detection tools include mass spectrometry (MS), UV-visible absorption (UV-Vis), infrared (IR) absorption, nuclear magnetic resonance spectroscopy (NMR), and atomic emission spectroscopy (AES).

The present disclosure seeks to exploit the simplicity and accessibility of the methods presented herein. High-performance detection methods such as MS and NMR require bulky and extremely expensive apparatuses. In the methods presented herein, analyte detection and identification requires only a Raman spectrometer, which is comparatively inexpensive, with portable and handheld systems available. SERS gives far more detailed information than UV-Vis, and it is far more sensitive than IR. Many of the separation technologies listed above require significant resources, including time-consuming preparations and specialized equipment. In contrast, the only equipment required by the methods presented herein is nanopaper and a beaker, test tube, or devices disclosed herein, and PC development only takes a few minutes. In combination, PC-SERS on nanopaper substrate is a much more accessible method than many of the technologies currently in use. This improvement over the other high-performance detection methods can prove beneficial to law enforcement, first responder, or parcel handlers for the detection of fentanyl, or fentanyl-related compounds.

The present disclosure demonstrates that the nanopaper presented herein offers multiple functions. In addition to the functions of enhanced detection and paper chromatograph, the nanopaper can serve as a vacuum filter paper to collect and concentrate suspicious substances dispersed in the air or spread on solid surfaces with different materials, including carpet, wood, and cloth. Therefore, sample collection, separation, and enhanced detection can be conducted in a single piece of nanopaper. The methods presented herein can minimize risks of exposure to fentanyl or other threat chemicals and reduce human errors during the material collection and transfer.

The present disclosure confirms that the nanopaper synthesis technique presented herein results in depositing a dense layer of silver particles onto the surfaces of glass microfiber filters, and it is further confirmed that this layer imparts large SERS signal enhancement relative to normal Raman scattering. The present disclosure seeks to demonstrate the capability by using PC-SERS with nanopaper to separate and identify an artificial mixture of dyes. The present disclosure further demonstrates an application of this technology by using the methods of the present disclosure to analyze and compare the carotenoid composition of several natural products.

In some embodiments, the nanopaper described herein can be utilized by researchers as a convenient SERS substrate, or utilized to develop specific assays and sold in combination kits for the detection of chemicals, such as, fentanyl. In particular embodiments, the present disclosure demonstrates the potential uses in drug or toxin testing, biochemical reactions analysis, and food safety testing. In some embodiments, the present disclosure seeks to demonstrate the potential use in identifying fentanyl with high accuracy.

### Working Examples

Reference will now be made to more specific embodiments of the present disclosure and data that provides support for such embodiments. However, it should be noted that the disclosure below is for illustrative purposes only and is not intended to limit the scope of the claimed subject matter in any way.

### Nanoplasmonic Paper Substrate

As discussed in brief above, surface-enhanced Raman scattering (SERS) is a powerful analytical tool which enables the detection and identification of analytes adsorbed on nanostructured noble metals. However, SERS analysis of complex mixtures can be challenging due to spectral overlap and interference. In the present disclosure, methods to simplify the identification of mixed-analyte samples by coupling SERS detection with chromatographic separation on a nanoplasmonic paper substrate are demonstrated. This “nanopaper” substrate is a silver-coated glass microfiber filter paper which possesses large SERS enhancement and can serve as a stationary phase in paper chromatography. Nanopaper is easily synthesized using the silver mirror reaction, making it a highly accessible technology. As shown herein, nanopaper was successfully used as a combined paper chromatography-SERS (PC-SERS) substrate in the separation and identification of mixed organic dyes. It was further employed to separate and identify lycopene and β-carotene in commercial food products, demonstrating the versatility and utility of nanopaper in the identification of complex mixtures.

Raman spectroscopy is a valuable detection tool because of its fingerprint identification of molecules, allowing for multiplexed analyte detection. Surface-enhanced Raman scattering (SERS) offers an improvement over normal Raman by increasing signal strength to allow detection of extremely dilute species. Furthermore, the advent of portable Raman spectrometers enables SERS for on-site work in diverse disciplines such as law enforcement forensics, environmental quality testing, food and drug testing, and point-of-care medical testing.

SERS is a near-field effect which greatly enhances the Raman scattering signal near nanostructured metal surfaces, primarily due to the localized electric field enhancement at such interfaces. The degree of enhancement is highly dependent on substrate composition and morphology; thus, the design of the SERS substrate helps in obtaining good results. Diverse classes of SERS substrates have been developed for sensing applications, including nanoparticle sols, chemically roughened metal surfaces, and solid supports modified with metal films. However, the fabrication of current SERS substrates is often tedious and relatively costly, which limits the accessibility of SERS substrates.

Another challenge in SERS identification, interpretation of spectral data, is common across spectroscopic methods. Depending on the sample, spectral interpretation can range from straightforward to highly challenging. As the number of chemical species in a sample increases, SERS peaks are more likely to overlap, confounding features. Peak fitting procedures can help resolve overlapping signals, but such treatments are often highly sensitive to user input. More sophisticated statistical methods, such as principal component analysis or hierarchical cluster analysis, may also be used to resolve a complex spectrum into its contributing component spectra.

These methods are useful, but require large data sets and reference libraries to be successful. A different approach to simplifying spectral data is to couple spectral analysis with a pre-separation procedure. This strategy enables collection of multiple simple spectra rather than a single complex spectrum in a process analogous to other common separate-and-then-detect methods like liquid chromatography-mass spectrometry (LC-MS). The combination of separation with molecular detection is a synergistic one. The separation process alone differentiates analytes but usually cannot uniquely identify them. In contrast, SERS spectra contain the information necessary for molecular identification, but can be challenging to interpret for mixed analytes. Prior methods have successfully coupled SERS with various separation methods, such as liquid chromatography (LC-SERS), thin layer chromatography (TLC-SERS), and Western blot, resulting in improved sensing performance. However, these methods require specialized instrumentation and would be difficult to perform in a non-laboratory setting.

A simpler and more accessible choice for separation is paper chromatography (PC), which is a well-established method for the separation of biological analytes. In PC, analytes are deposited on a paper or paper-like substrate and a mobile phase is eluted through the substrate via capillary action. Separation occurs based on phase partitioning (solubility in the mobile phase) and affinity (interaction between analyte and substrate). While other chromatographic methods can result in separation, the simplicity and accessibility of PC nevertheless makes it a useful tool. Achieving PC-SERS requires a substrate that functions as a PC stationary phase while also providing Raman enhancement for SERS identification. Previous studies have synthesized PC-SERS substrates by modifying porous solid surfaces with noble metals using various methods, including inkjet printing nanoparticle ink onto cellulose paper, thermally evaporating and annealing silver films on cellulose paper, impregnating chromatography paper with silver-silica core-shell nanoparticles, and modifying glass fiber sheets with molybdenum disulfide followed by reduction of chloroauric acid. These PC-SERS substrates were successfully used to separate and detect various substances such as organic dyes and narcotic drugs; however, each required multiple complicated synthetic steps to produce, limiting accessibility for widespread use.

The present disclosure utilizes the silver mirror reaction with commercial glass microfiber filter paper in a facile protocol to synthesize nanoplasmonic paper (called nanopaper). Nanopaper is a glass microfiber filter paper decorated with a dense layer of silver nanoparticles, which possesses large SERS enhancement. The well-known mirror reaction offers a rapid, simple, robust, and effective way to form silver deposits on a large amount of glass fiber papers in a single batch reactor. The granular nanostructured silver film can be created by controlling the reaction conditions, offering large SERS enhancement to silica substrates. Nanopaper proved to be a viable PC-SERS substrate capable of the separation and identification of mixed organic dyes. Furthermore, the facile, scalable synthesis requires very few materials and little expertise, allowing this separation-detection technology to have broad accessibility across multiple research disciplines. In order to demonstrate the versatility of nanopaper, the present disclosure further utilizes the nanopaper to analyze the carotenoid profile in commercially available food products, including carrot and tomato juices and vegetable juice blend.

Materials.

Potassium hydroxide pellets, ammonium hydroxide solution (28-30%), rhodamine 6G (95%), crystal violet (90%), toluene (99.5%), dichloromethane (99.5%), anhydrous magnesium sulfate, and lycopene (90%, from tomato) were purchased from Sigma-Aldrich. β-Carotene (97%) was acquired from Enzo Life Sciences. Acetone (99.8%), hexanes (99.9%), 2-propanol (99.9%), methanol (99.9%), acetic acid (glacial), silver nitrate (99.9995%), and Whatman binder-free glass microfiber filters (grade 934-AH, 110 mm circles) were purchased from Fisher Scientific. D-Glucose (99.5%), methyl orange (ACS grade), ethanol (200 proof), and cresol red (pure, indicator grade) were purchased from VWR International. Before use, the lycopene solid was rinsed with methanol and allowed to dry. All other materials were used directly without further purification. Carrot juice (Bolthouse Juice Products LLC), tomato juice (Campbell Soup Company), and V8® Original juice blend (Campbell Soup Company) were purchased from a local market.

Nanopaper Synthesis.

Nanopaper PC-SERS substrate was synthesized by performing the silver mirror reaction in the presence of glass microfiber filters. Tollens' reagent was prepared according to a reported protocol by adding potassium hydroxide (3.2% aqueous) to 40 mL of 2% silver nitrate solution until a brown precipitate formed, followed by dropwise addition of ammonium hydroxide (˜30%) until the solution became colorless and transparent. Additional silver nitrate was added until a yellow color persisted, and finally diluted ammonium hydroxide (6%) was added until the solution again became transparent. Immediately after its preparation, a stack of four glass microfiber filters was immersed in the Tollens' reagent solution. To initiate the mirror reaction, 40 mL of 35% aqueous D-glucose solution was combined with 20 mL methanol and then added to the Tollens' reagent, shaking to mix thoroughly. The glass microfiber filters were left in this reaction mixture for 1 h, then removed and washed with copious amounts of water and 2-propanol. The resulting nanopaper was stored immersed in 2-propanol, protected from light.

Carotenoid Extraction from Vegetable Juices.

To extract carotenoids from the vegetable juices (carrot juice, tomato juice, and V8® Original juice blend), an adapted, reported protocol, was utilized. Briefly, 5 mL of juice was added to 5 mL of 1:1 hexane:acetone (v/v). The mixture was vortexed and sonicated, then centrifuged. The organic phase was then saponified with potassium hydroxide solution (40%, in methanol) at 56° C. for 45 min. The extract was then washed with 10% aqueous magnesium sulfate, filtered to remove solids, washed 3 times with water, dehydrated over anhydrous magnesium sulfate, and filtered. This left an extract containing water-insoluble, nonsaponifiable organics, including carotenoids. The extract was concentrated in a rotary evaporator, reconstituted in a minimal volume of dichloromethane, and stored at −20° C., protected from light.

PC-SERS with Nanopaper.

Nanopaper samples were removed from storage, cut into strips, and air-dried. To prepare a nanopaper strip for PC, ˜0.25 μL of analyte solution was spotted by micropipette near the bottom edge and allowed to dry. Then, the sample was loaded into a beaker containing the mobile phase, taking care to avoid direct contact between the mobile phase and the analyte spot. The mobile phase used for the 4-dye mixture was 9:1 (v/v) toluene/acetic acid, and the mobile phase used for carotenoid separation was absolute ethanol. When the solvent had migrated a sufficient distance up the nanopaper, the paper was removed, the location of the solvent front was marked, and the sample was oven-dried before SERS data collection.

Raman measurements were collected using a Thermo Scientific DXR Raman microscope (Thermo Fisher Scientific, Inc.) equipped with 780 nm diode laser excitation, a 10× objective lens, a Rayleigh rejection filter, a high-resolution (˜2 cm⁻¹) diffraction grating, and a CCD detector. Spectral intensity was acquired in the range of 400-1800 cm⁻¹. Each spectrum was acquired at 1 mW laser power with 5 accumulations of 1 s integration time. Developed chromatograms were measured at regular intervals (0.25 mm for 4-dye mixture, β-carotene, and lycopene, and 0.50 mm for juice extracts) on a line parallel to solvent flow to construct SERS maps. The OMNIC™ software package was used to smooth and baseline correct spectra; all spectra were then scaled with respect to the nanopaper background signal at 900 cm⁻¹ to account for sample roughness, which caused small variations in overall spectral intensity as the sample surface deviated above or below the microscope focal plane. To construct component intensity profiles from the processed SERS maps, scaled intensity was determined for characteristic wavenumbers and averaged to achieve 1 mm spatial resolution. Each intensity profile relates the results of a single PC-SERS experiment.

Nanopaper Synthesis and Characterization.

The nanoplasmonic PC-SERS substrate, referred to herein as “nanopaper”, was designed with three criteria in mind: (1) it should be easily synthesized from readily available materials; (2) it should facilitate the separation of compounds based on their physical properties; and (3) it should possess sufficient SERS enhancement to allow detection of analytes that would otherwise be difficult to identify.

Nanopaper was synthesized by modifying binder-free glass microfiber filters. The glass filters serve as a mechanically strong support that is relatively chemically inert. Their structure of intertwined glass fibers is also conducive to capillary action, which drives mobile phase elution in paper chromatography. Because glass does not provide the SERS enhancement necessary to a PC-SERS substrate, the mirror reaction was employed to impregnate the glass fiber filters with silver, the element with the largest SERS enhancement. In this simple and straightforward reaction, glucose was used to reduce diamine silver(I), resulting in elemental silver particles which precipitated on the glass fiber surfaces. The silver mirror reaction was chosen for its simplicity and familiarity, as well as its effectiveness in coating glass surfaces with silver. All synthetic steps, including reagent preparation, reaction, and purification, could be completed in a large batch reactor in less than 2 h.

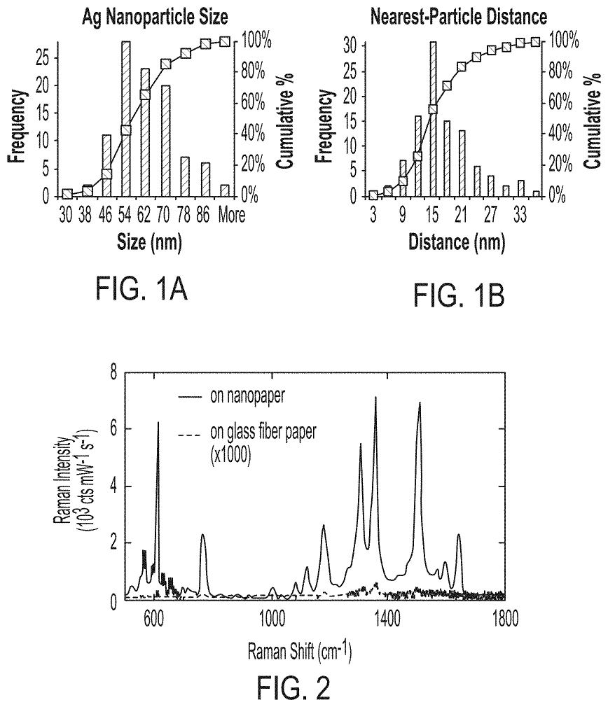

Nanopaper synthesis results were analyzed by visual appearance, SEM, and SERS. During synthesis, the color of the glass microfiber filters changed from white to dark gray/brown, indicating the deposition of silver particles. This was confirmed by SEM, which showed a dense layer of nanoparticles covering the glass fiber surfaces, with average particle size below 100 nm, and an average nearest-particle distance less than 20 nm in most samples (FIG. 1A and FIG. 1B). The morphology of the silver coating was not observed to change after performing paper chromatography. The SERS activity of the nanopaper was determined by comparing the Raman signal of rhodamine 6G (R6G) adsorbed on unmodified glass fibers against the SERS signal of R6G adsorbed on nanopaper (FIG. 2). The result showed large signal from the nanopaper sample while the plain glass fiber sample could barely be detected, indicating that the silver coating of the nanopaper provided SERS enhancement. The analytical enhancement factor was approximated by calculating the peak intensity ratio of the SERS and normal Raman samples. For the 612 cm⁻¹ peak of R6G in FIG. 2, the enhancement factor was 1.15×10⁵. To further verify nanopaper reproducibility, the enhancement factor was calculated for three different batches of nanopaper, synthesized on different days, using a different dye solution (0.4 mM crystal violet; CV). For the 417 cm⁻¹ peak of CV, effective enhancement factors of the three different batches were 1.15×10⁵, 1.17×10⁵, and 1.05×10⁵. The consistent enhancement between nanopaper batches demonstrated the reproducibility.

Overall, the nanopaper exhibited good durability throughout experiments. Changes in physical properties and SERS activity were not observed during the various drying, elution, and SERS measurement steps of the PC-SERS protocol, or after brief periods of oven drying (<1 h at 75° C.). After extended time (>1 day) on the benchtop or in the oven, nanopaper lost some SERS activity, became hydrophobic, and experienced a slight change in color; thus, all SERS measurements of nanopaper samples were performed within the same day the sample was removed from storage. However, when stored in isopropanol and protected from light and air, no noticeable degradation of the nanopaper over a period exceeding 2 months was observed.

PC-SERS Performance.

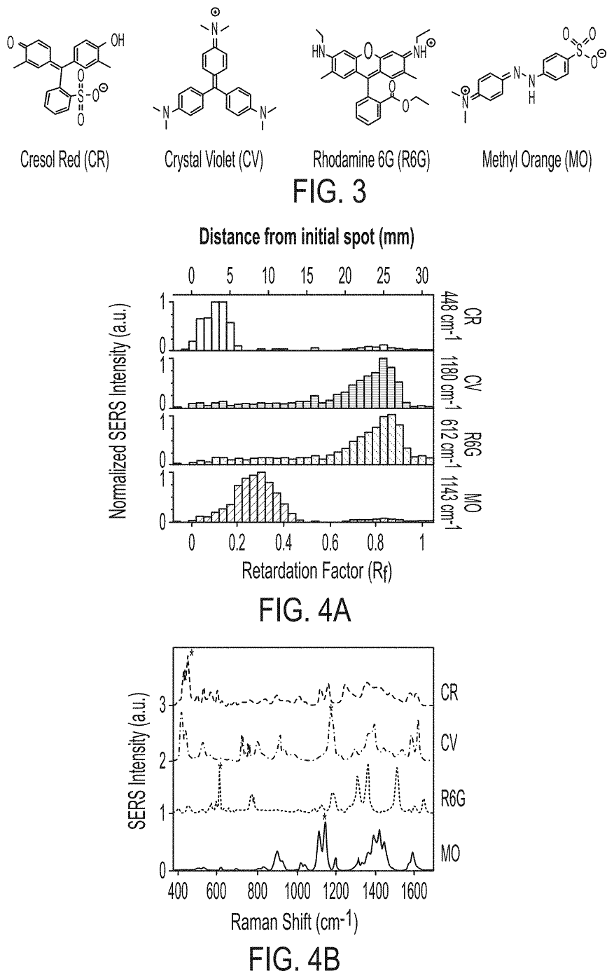

Nanopaper performance was tested by implementing the PC-SERS protocol on a mixture of four organic dyes: R6G, cresol red (CR), crystal violet (CV), and methyl orange (MO). FIG. 3 illustrates the dye structures. The dye mixture (4×10⁻⁴ M in each dye, aqueous) was spotted on nanopaper and eluted with a mobile phase of 9:1 (v/v) toluene:acetic acid. During elution, dye spots were observed to migrate upward, separating into three faintly colored bands. These bands faded upon drying the nanopaper, becoming invisible, or barely visible, and making the developed chromatogram challenging to interpret visually. The SERS mapping results helped to interpret the PC separation, showing distinctly different spectral patterns across the length of the chromatogram. The location of each dye was determined based on the SERS intensity profile of its characteristic peak (FIG. 4), and expressed in terms of a retardation factor (R_{f}). R_{f} is the fractional migration distance from the initial spot relative to the total migration distance of the mobile phase, and is calculated by Equation 1, shown below.

$${R}_{f} = \frac{{x}_{i} - {x}_{0}}{{x}_{front} - {x}_{0}} \qquad Equation 1$$

In Equation 1, shown above, xᵢ, x₀, and x_{front} are the locations of the ith measurement, the center of the initial spot, and the solvent front, respectively. The intensity profiles identified the composition of the three bands observed during PC: the first band contained CR with a maximum intensity at R_{f}≈0.11, the middle band contained MO with a maximum intensity at R_{f}≈0.30, and the third band contained both CV and R6G with maximum intensities at R_{f}≈0.84 and R_{f}≈0.87, respectively. The primary factor in elution distance appears to be solubility in the mobile phase used, based on the observation that the dyes eluted in the order of increasing solubility in 9:1 toluene:acetic acid (CR<MO<CV/R6G).

The separation resolution of this PC-SERS result was not as good as some other chromatographic techniques, such as TLC and LC. Co-elution of CV and R6G occurred, and some band spreading, or streaking, was present for all species. It is believed this could be improved through protocol optimization, for example, by functionalizing the surface to modify hydrophobicity or by selecting a better mobile phase. However, the separation was sufficient to easily distinguish spectral constituents by SERS, making such optimization superfluous for this case.

PC-SERS Demonstration with Extracted Carotenoids.

To further demonstrate the utility of PC-SERS with nanopaper, the technique was applied to identify β-carotene and lycopene in food products, including tomato juice, carrot juice, and V8® original juice blend. β-carotene (highly enriched in carrot) and lycopene (mainly found in tomato) are two major carotenoids that can serve as precursors of retinol and bioactive antioxidant compounds, playing an important role in human health. Due to the chemical similarity of β-carotene and lycopene, traditional analyses of carotenoids combine high-performance liquid chromatography (HPLC) with different detection methods (e.g., UV/vis or mass spectrometers) to improve specificity. The requirement of special instruments limits the accessibility of carotenoids analysis; thus, the nanopaper technique offers an alternative solution for food industry.

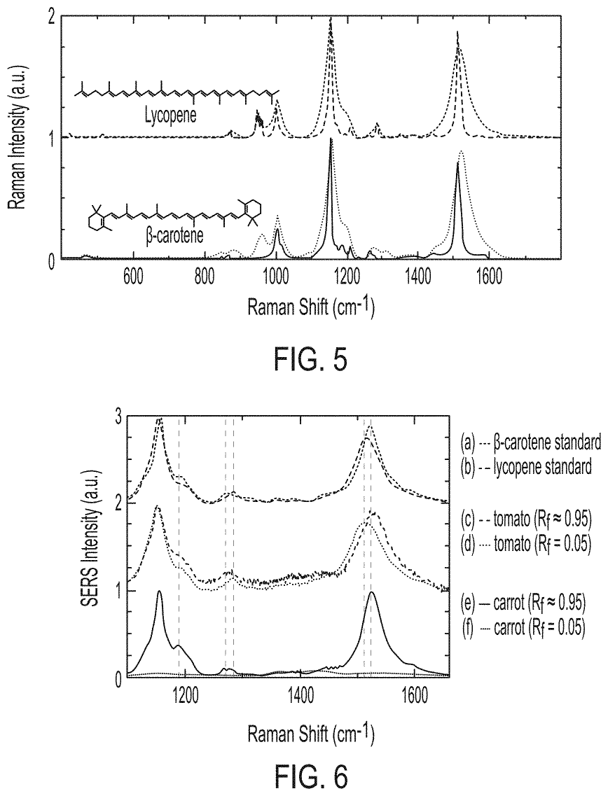

The SERS spectra of lycopene and β-carotene were highly similar, exhibiting prominent peaks at ˜1154 cm⁻¹ and ˜1520 cm⁻¹ (FIG. 5). The largest spectral differences between the two are fluctuations in the shoulder peak near 1200 cm⁻¹ and in the doublet near 1300 cm⁻¹, and an intensity and wavenumber shift of the peak near 1520 cm⁻¹ (FIG. 6). To analyze the carotenoid profile of carrot juice, tomato juice, and V8® juice blend, PC-SERS was performed on organic extracts from each product. For comparison, the PC-SERS protocol was also applied to lycopene and β-carotene references. Ethanol was used as the mobile phase for each sample. During elution, the β-carotene reference was seen to migrate with the solvent front as a faint yellow band, whereas the lycopene reference did not visibly move from its initial spot. Since the ethanol solubility of β-carotene is higher than that of lycopene, analyte partitioning in the mobile phase (solubility) is probably the a factor in this PC separation.

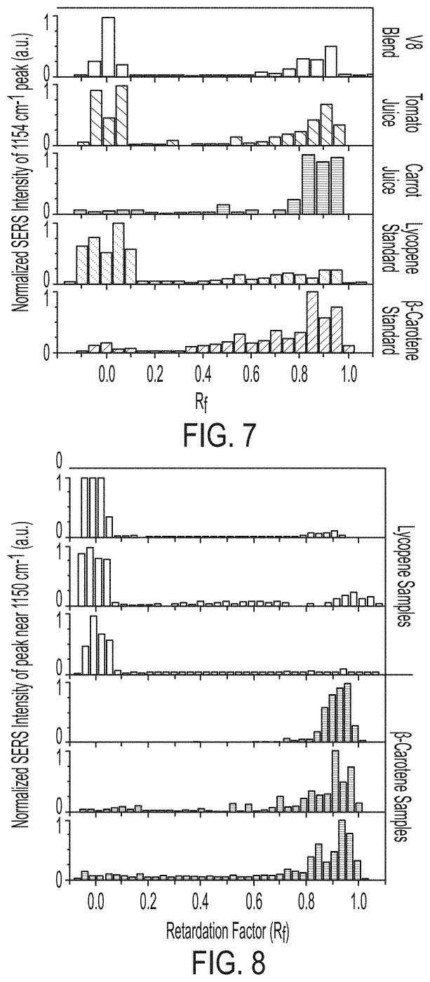

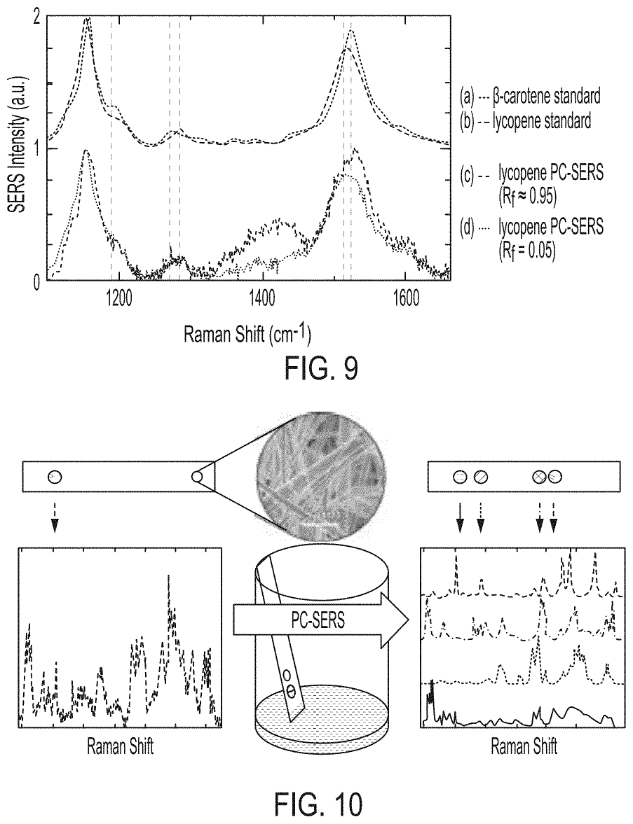

SERS intensity profiles based on the major carotenoid peak observed at 1154 cm⁻¹ (FIG. 7) revealed a strong spectral signal near R_{f}=0 on the lycopene sample, and at 0.8<R_{f}<1 on the β-carotene sample. Three additional PC-SERS replicates were performed for lycopene and β-carotene references to ensure repeatability (FIG. 8), and each replicate's results were consistent with those found in FIG. 7. Some weak signal was observed in the lycopene reference sample at 0.5<R_{f}<1, which is believed to be the result of β-carotene contamination in the lycopene standard (FIG. 9). The SERS intensity profile for carrot juice extract showed localization of carotenoid peaks mostly at 0.8<R_{f}<1. The tomato juice extract and the V8® extract both showed localization centered near R_{f}=0 and, to a lesser extent, at 0.8<R_{f}<1.

The results of FIG. 7 imply that the major carotenoid identified in carrot juice was β-carotene, while tomato juice and V8® juice blend contained both lycopene and β-carotene. This was confirmed by examining individual spectra within SERS maps of the carrot and tomato extracts (FIG. 6). The tomato spectrum collected from R_{f}=0.05 exhibits a greater similarity to the lycopene reference spectrum, whereas the spectra at R_{f}=0.95 are more similar to the β-carotene reference spectrum for both carrot and tomato juice extracts. The V8® PC-SERS results show the same trend.

In conclusion, the present disclosure illustrates successfully synthesized PC-SERS substrates, nanopaper, by performing the commonly known silver mirror reaction in the presence of glass microfiber filters. FIG. 10 illustrates a nanopaper substrate PC-SERS illustration. The synthesis is simple and scalable, resulting in a stable product that can be used as both a PC separation stationary phase and a SERS substrate. The utility of the nanopaper substrate was demonstrated by performing the separation and subsequent SERS identification of mixed organic dyes, as well as extracts from natural products. This demonstration showed the synergistic power of coupling PC separation with SERS detection; the PC separation simplified the SERS identification of mixed analytes, while the SERS identification simplified the interpretation of the PC separation. The low-cost, disposable, and portable nanopaper can serve as a versatile technique for the identification of chemical and biological compounds within complex matrices.

### Applications and Advantages

The ability to quickly detect and identify unknown substances, especially those suspected to be narcotics, is critical for all law enforcement agents, first responders, and parcel handlers. When unknown substances are found during a search, for example, it is critical to determine what the unknown substances are. As such, a further objective of the present disclosure is directed to a method to collect and identify chemical dust through vacuum filtration followed by surface-enhanced Raman scattering (SERS) spectroscopy. Both operations are performed using a single SERS-active filter substrate, or nanopaper, as described in detail above. In some embodiments, the collection and identification can include identifying various opioids or synthetic opioids. In particular embodiments, described in further detail below, the nanopaper can further include 1-butanethiol to detect trace amounts of fentanyl.

As previous discussed in the preceding sections, Raman scattering possesses attractive advantages for use in chemical detection. It provides fingerprint molecular identification, it can simultaneously detect multiple analytes and, due to the availability of portable and handheld systems, it is suitable for laboratory or fieldwork. However, Raman spectroscopy suffers from inherently weak signal, making it difficult to use for trace detection, for example, detection of trace amounts of fentanyl. SERS is a modification of Raman spectroscopy which greatly amplifies the signal of molecules captured onto an engineered solid substrate, and it maintains the advantages of normal Raman detection while allowing much more sensitive detection. The greatly enhanced signal from SERS comes at the cost of additional sample preparation and the necessity for a well-designed SERS substrate. Therefore, to enable SERS applications, these aspects should be simplified to the greatest extent possible.

A nanopaper trace chemical dust detector, described herein, simplifies sample preparation by enabling sample collection directly onto the SERS substrate. This is achieved by using, for example, a SERS-active vacuum filter as a substrate. This nanoplasmonic filter paper substrate, referred to as nanopaper, is composed of glass microfiber filters modified with a dense layer of silver nanoparticles which impart SERS activity, as discussed in detail above. It can be simply synthesized via the silver mirror reaction (also called the Tollens' reaction) performed on fiberglass filters.

In various embodiments, a nanopaper sample is loaded into a vacuum so that it obstructs the flow, allowing air to pass but filtering any solid particles. After collection, the nanopaper is measured by Raman scattering. In some embodiments, a drop of solvent, such as water or ethanol, could be added before measurement to improve the analyte contact with the nanopaper substrate.

The versatility illustrated in the present disclosure is of particular note, as the nanopaper described herein can detect trace chemicals suspended in air or deposited on virtually any surface including cloth, paper, carpet, and the like. Furthermore, nanopaper can be implemented on virtually any vacuum system, for example, a portable vacuum, and for applications in which the safety of potential analytes is a particular concern, it could even be outfitted for use with robotic vacuum cleaners for remote detection, for example, identifying unknown chemicals in high quantities that are suspected of being toxins, for example, fentanyl.

The potential applications are manifold. The nanopaper and systems of use described herein is well suited to fields such as law enforcement and forensics, where it could find use detecting explosives, illegal drugs, or other chemical agents, without exposing personnel to hazardous conditions, for example, high potency drugs such as, but not limited to, fentanyl. The nanopaper and systems of use described herein can also be used in such fields as environmental science and air quality testing.

The present disclosure represents the first Raman detector with both functions of chemical detection and dust collection. In addition to dust collection and SERS detection, the nanopaper possesses chromatograph function, which allows separation of trace chemical from the complex background matrix. As detailed below, a drop of methanol can separate R6G from garden soil, enhancing the detection signals. The surface of nanopaper can be easily modified using thiol chemistry, and surface modifications can improve the adsorption of target chemicals, improving the detection sensitivity.

As discussed in further detail below, in a particular embodiment, nanopaper modified by 1-butanetiol enhances the signal of fentanyl. The fabrication of nanopaper is simple and low-cost. Moreover, nanopaper is disposable and can be adapted to any commercial vacuum cleaner, including robotic vacuum cleaners. This particular application can prove beneficial for law enforcement, first responders, parcel handlers, and various security checkpoints, such as customs and boarder protection to eliminate the traffic of illegal narcotics, such as, but not limited to, fentanyl.

### Multi-Functional SERS Substrate: Collection, Separation, and Identification of Airborne Chemical Powders on a Single Device

Due to its extreme sensitivity and fingerprint specificity, surface enhanced Raman spectroscopy (SERS) is a powerful tool for substance identification. Developments in portable low-cost SERS substrates and handheld Raman spectrometers enable SERS analysis at sample origin, with great potential benefit to fieldwork applications in numerous disciplines. The present disclosure illustrates a procedure which incorporates sample collection, isolation, and SERS identification of airborne solids on a single inexpensive substrate. This procedure, vacuum filtration-paper chromatography-SERS (VF-PC-SERS), utilizes a porous filter paper decorated with plasmonic nanoparticles, herein referred to as nanopaper, and discussed in detail above. The porous fiber structure facilitates both the vacuum filter powder capture and the isolation of components by paper chromatography, while the nanoplasmonic coating enhances Raman signal. One potentially high-impact application of VF-PC-SERS is field analysis of hazardous or illicit materials. This disclosure demonstrates VF-PC-SERS using powdered rhodamine 6G (R6G) dispersed in air, resulting in 100% detection accuracy at R6G levels as low as 0.6 mg/m³. Analysis of R6G contaminated with topsoil or lactose resulted in specific identification of R6G in powder mixtures containing as little as 0.1 wt. % R6G. This disclosure demonstrates the feasibility of VF-PC-SERS as a safer procedure to identify hazardous substances at the point of sample origin.

Field identification of chemicals is vital to many applications, especially in situations which involve screening potentially hazardous substances, such as explosive or toxic chemicals. Hazard identification faces the added challenge of safely collecting and processing samples. For example, field analysis of seized narcotics by law enforcement agents can result in exposure and potential overdose. The presence of other materials in impure field samples often means that samples must be purified before analysis. This further increases the danger because each operation performed on the sample adds another potential for toxic exposure. Current methods for the identification of suspicious or hazardous substances usually involve transporting samples to a laboratory for analysis with ELISA and GC-MS or field-testing with colorimetric assays, which have low specificity and could require the collection of potentially lethal sample quantities. Thus, there is a need for an analytical device which enables the identification of unknown substances at the point of sample origin, while minimizing the risk of exposure to hazardous materials.

Raman spectroscopy is an ideal alternative for chemical detection because it can provide fingerprint molecular identification of multiple species simultaneously. Furthermore, portable and handheld Raman instrumentation is available, making it suitable for either laboratory or fieldwork. However, despite its high specificity, standard Raman spectroscopy has limited use in trace detection due to its inherently weak signal which is easily overwhelmed by the fluorescence background, especially in dilute or complex samples. This weak signal can be remedied by the use of surface enhanced Raman spectroscopy (SERS), which utilizes an engineered solid substrate composed of noble metal nanostructures to amplify the Raman signal by a factor of 10²-10¹⁰. The degree of enhancement depends on the composition and geometry of the substrate, as well as the target molecule's proximity to the substrate surface. Currently, most SERS substrates are fabricated as nanostructure films formed on rigid solid supports by various methods, including self-assembly, templated assembly, and nanolithography. Previous studies have demonstrated the ability of SERS to detect trace levels of toxic narcotic compounds, even when mixed with other drugs.

The near unparalleled combination of sensitivity and specificity achieved by SERS enables a wide range of potential applications for trace chemicals analysis; however, the advantages of SERS are not without price. The need for an engineered substrate introduces additional materials, labor, and cost to the analysis and the need for intimate chemical contact between the substrate and the sample necessitates additional sample preparation. On rigid substrates, SERS tests are generally performed by collecting and dissolving the sample, depositing the solution on the substrate, washing, drying, and finally acquiring spectra. Background fluorescence and spectral interference in complex samples may necessitate additional separation procedures before depositing the sample on the SERS substrate. This means that SERS analysis requires not only a substrate and a spectrometer, but also sample collection equipment, solvents, clean glassware, separations equipment, pipettes, and other laboratory paraphernalia. This is impractical for field analysis; ideally, a field-ready SERS assay would require only a sample, a substrate, and a spectrometer. Each additional apparatus used increases the risk of a human error resulting in hazard exposure or sample contamination.

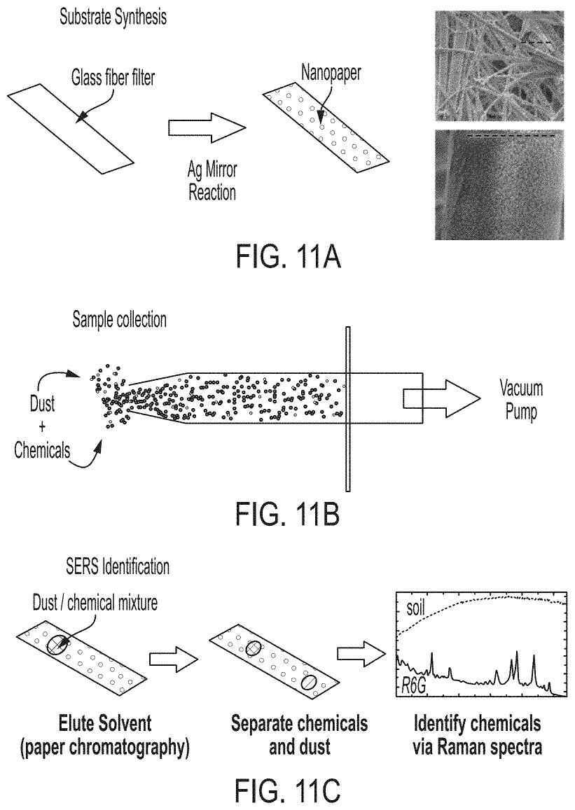

A strategy to simplify separation and identification of complex mixtures by performing paper chromatography (PC) directly on a SERS substrate in a method called PC-SERS (FIG. 11) is demonstrated. The substrate, called nanopaper, is simple and inexpensive to produce; it is composed of a glass microfiber filter coated with a dense layer of silver nanoparticles which impart SERS enhancement. By depositing chemical mixtures on one end of a nanopaper strip, eluting the strip with a solvent, and measuring the SERS spectra at different locations on the strip, mixture components can be unambiguously identified. The effectiveness of this strategy was demonstrated for mixtures of organic dyes and for carotenoids in vegetable extracts, as detailed above. The separation protocol occurs on the SERS substrate; no additional equipment or reagents are necessary beyond a small quantity of solvent. Compared to assays that require off-substrate sample cleanup, the use of nanopaper and PC-SERS in field analysis could improve portability and decrease cost. This strategy could also reduce hazardous exposure because sample manipulation only occurs when immobilized on a solid surface.

Nanopaper, in addition to its functions of separator and signal enhancer, can be used as a sample collector. Nanopaper pores can capture solids and the microfiber structure easily wicks up liquids, allowing it to be used as a swab or dipstick. A more interesting application, however, is to employ nanopaper as a filter to capture analytes. Porous SERS substrates can be used as filters for isolation and SERS identification of trace substances in liquid, including aqueous tyrosine, pesticides, organic dyes, and milk adulterants (melamine, sodium sulfocyanate, dicyandiamide), among others. While these methods offer valuable strategies for trace analysis of liquid samples by SERS, filtration is utilized for purification by membrane extraction rather than for sample collection.

Disclosed herein is the use of nanopaper for solid powder collection, purification, and identification using vacuum filtration-paper chromatography-SERS (VF-PC-SERS). To accomplish VF-PC-SERS, a nanopaper filter is installed on a vacuum pump and used to collect solid particles dispersed in the surrounding air (FIG. 11). After collection, solvent is eluted through the nanopaper to achieve close contact between the sample and the SERS substrate and, in the case of impure samples, to achieve chromatographic separation. Eluted samples are then analyzed using SERS. Experiments were performed in a laboratory setting with a benchtop Raman spectrometer, but the procedure could be easily performed in the field; in the presence of an airborne powder, the only necessary materials are nanopaper, a vacuum cleaner, a small volume of solvent, and a portable Raman spectrometer.

Materials.

Potassium hydroxide pellets, ammonium hydroxide solution (28-30%), rhodamine 6G (95%), and fentanyl (1.0 mg/mL in methanol) were purchased from Sigma-Aldrich. 2-propanol (99.9%), methanol (99.9%), silver nitrate (99.9995%), and binder-free glass microfiber filters (Whatman grade 934-AH, 110 mm circles) were purchased from Fisher Scientific. D-glucose (99.5%) and D-lactose (99.9%) were purchased from VWR International. Garden soil was purchased at a local hardware store.

Nanopaper Fabrication and Characterization.

Nanopaper was fabricated as described above, without modification. Briefly, an aqueous solution of ammonia and silver nitrate was activated by mixing with potassium hydroxide; then, glass microfiber filters were fully submerged in the silver solution. The silver mirror reaction was initiated by rapidly adding an aqueous glucose solution to the reaction vessel and shaking for several minutes. After the reaction was complete, nanopaper sheets were rinsed thoroughly with water and with 2-propanol, then stored immersed in 2-propanol. Before use in VF-PC-SERS experiments, nanopapers were air dried and cut into 1 cm×2 cm rectangular sample strips.

Vacuum Filtration Experiments.

To remove water from the powder samples and allow them to more easily disperse into the air, R6G, D-lactose, and topsoil were dried in a nitrogen-purged oven at 70° C. The mass of each powder was measured hourly until no mass change was observed, which occurred after about 6 h. Topsoil was sifted to remove wood fragments and other objects larger than several millimeters. After drying, a Beckmann Coulter Particle Size Analyzer LS 13320 was used to characterize the particle size distribution for each powder. Immediately prior to vacuum filtration experiments, powders were removed from the drying oven and weighed out into a petri dish. For experiments involving mixed powders, they were stirred until the sample appeared homogeneous.

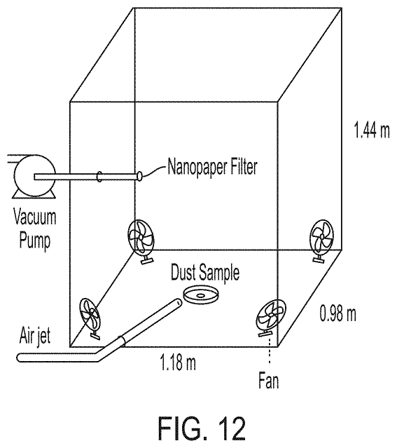

The collection chamber was constructed by draping polyethylene sheeting over a rectangular metal frame, with an electric fan placed in each corner to maintain air circulation (FIG. 12). A vacuum pump was connected to the chamber by inserting the intake tube through a small slit in the center of one chamber wall, and compressed air was delivered to the center of the chamber floor through a tube inserted at the floor of the adjacent wall. The total volume of the chamber was 1.67 m³. To set up vacuum filtration experiments, the vacuum pump was switched on and adjusted to a pressure differential of 50 kPa. A nanopaper filter strip was then placed over the vacuum inlet and a petri dish containing the powder specimen was placed in the center of the chamber floor. Then, the chamber was closed off and the electric fans were switched on. To initiate collection, the powders were dispersed into a dust cloud by pulsing a valve permitting compressed air to disperse the sample in the chamber. Vacuum filtration proceeded for either 60 s or 20 s, after which the fans were switched off and the nanopaper filter strip was removed for PC-SERS analysis.

Paper Chromatography-SERS Analysis.

After vacuum filtration, the bottom edge of each sample strip was dipped into methanol and eluted until the solvent front fully cleared the circular region where the vacuum line had been placed. The sample strips were then dried briefly in an oven at 70° C. and transferred to the spectrometer for SERS acquisition.

Raman measurements were collected using a Thermo Scientific DXR Raman microscope (Thermo Fisher Scientific, Inc.) equipped with 780 nm diode laser excitation, a 10× objective lens, a Rayleigh rejection filter, a diffraction grating (4.7-8.7 cm⁻¹ resolution), and a CCD detector. For experiments with 60 s VF duration, 50 spectra were acquired from a regular grid near or at the solvent front, using color to help select the grid location. In experiments with reduced (20 s) VF duration, spectra were instead acquired along the center of the nanopaper strip, without regard to visual cues. 5 accumulations of 1 s were used, and spectra were acquired at ≤1 mW laser power to avoid sample heating. Spectra were processed to remove artifacts and baseline contributions and to reduce spectral noise. After processing each spectrum, peak intensities were recorded for the R6G peak near 612 cm⁻¹, and the mean intensity was calculated for each VF-PC-SERS sample strip.

Nanoplasmonic Paper and SERS Measurement.

Nanopaper synthesis resulted in the formation of a dense layer of silver nanoparticles on the surfaces of the glass microfiber filter precursor. The silver nanoparticles caused the filter to change color from white to brown and imparted a strong SERS enhancement effect to the material. The nanopaper retained the filtering and chromatographic capabilities of the original glass fiber filters, and it demonstrated good stability and longevity when stored immersed in 2-propanol and shielded from exposure to light. It was found that removing the nanopaper from storage conditions did not cause the SERS enhancement to degrade in the timescale of the experiments (minutes to hours). A complete characterization of the nanopaper substrate is described in detail above.

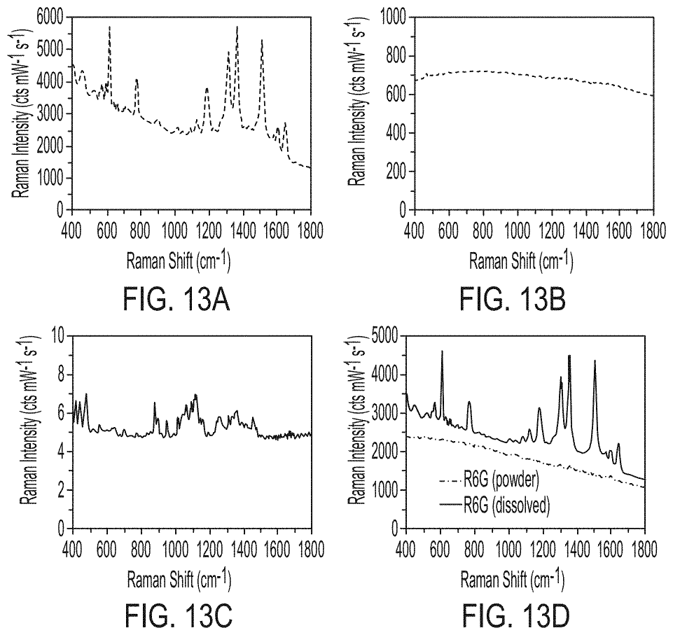

All vacuum filtration experiments were performed using solid R6G as the target substance, due to its low toxicity. The underlying purpose was to use a single substrate for collection and SERS enhancement, so powders, including R6G, lactose, and topsoil, were subjected to SERS analysis. While acquiring the SERS spectra of powders, it was found that particles deposited on the nanopaper filter had insufficient contact with the surface to benefit from signal enhancement (FIG. 13). The solid phase Raman spectrum of R6G was weak and dominated by fluorescent background. It was necessary to dissolve the powder, either by adding a drop of solvent or by employing paper chromatography, to obtain satisfactory SERS spectra. A variety of solvents could be used, but methanol was chosen for its quick evaporation and because R6G is easily soluble in methanol. Once dissolved on the substrate, R6G powder had a strong SERS signal (FIG. 13). The peak near 612 cm⁻¹ on the R6G SERS spectrum was strong and well defined, so this peak intensity was used to indicate the presence of R6G. Solid D-lactose and soil powders did not have strong Raman spectra (FIG. 13). Upon addition of solvent, D-lactose and soil still did not yield observable SERS spectra. This was because topsoil does not dissolve in methanol, and D-lactose dissolves only sparingly in methanol resulting in a lack of close contact between the powders and the plasmonic surface.

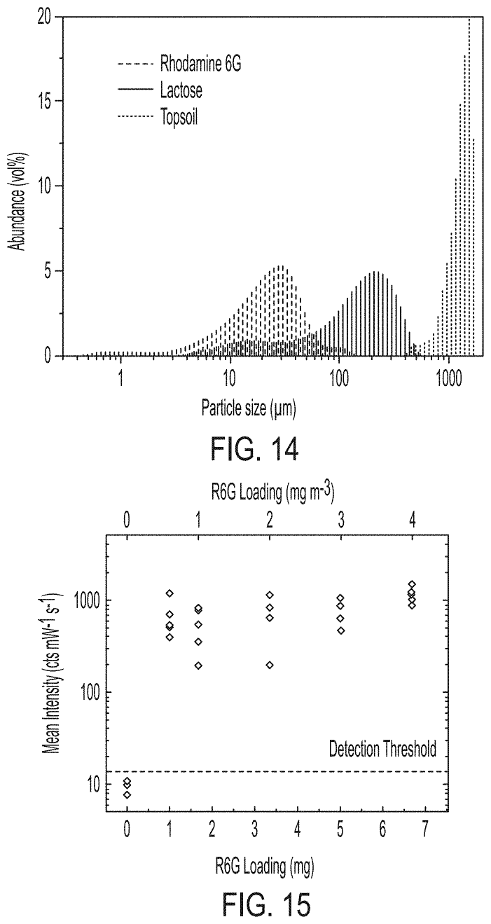

For sample collection by vacuum filtration to be effective, the filter pore size should be compatible with the particle size of the target powder. Therefore, R6G, D-lactose, and topsoil, were characterized by particle size distribution. The results showed that all samples had left-skewed size distributions (FIG. 14). R6G had the finest grain size, with a median diameter of 22.5 μm. D-lactose particles were larger with a median diameter of 158.1 μm. The topsoil contained some very large particles that were outside the range of the particle size analyzer; however, among particles that were within the instrumental range, the median diameter was 1380 μm. According to the manufacturer, the glass fiber filter that serves as the nanopaper precursor has a typical particle retention of 1.5 μm. Therefore, the nanopaper filter is capable of retaining all lactose and soil particles as well as >99% of the R6G.

Collection and Identification of Airborne Chemical Powders Via Vacuum Filtration.

To test the R6G detection capability of VF-PC-SERS, the protocol was implemented with varying amounts of R6G powder loaded into the collection chamber. Sample loadings of 6.7, 5, 3.3, 1.7, and 1 mg (equivalently 4, 3, 2, 1, and 0.6 mg/m³) were tested with a VF duration of 60 s, with 5 experimental replicates for each loading. After each experiment, a negative control was performed with no R6G to determine whether residual R6G would affect the following experiment. Upon removal from the vacuum filtration chamber, examination of the nanopaper sample strips by eye did not show the presence of R6G; however, particles were visible by microscope and upon elution with methanol, a bright pink band migrated with the solvent front. SERS analysis at the solvent front of these samples showed an identifiable peak near 612 cm⁻¹, confirming the presence of R6G. None of the negative control samples exhibited a colored band during elution, and none showed any reliable R6G peaks (FIG. 15).

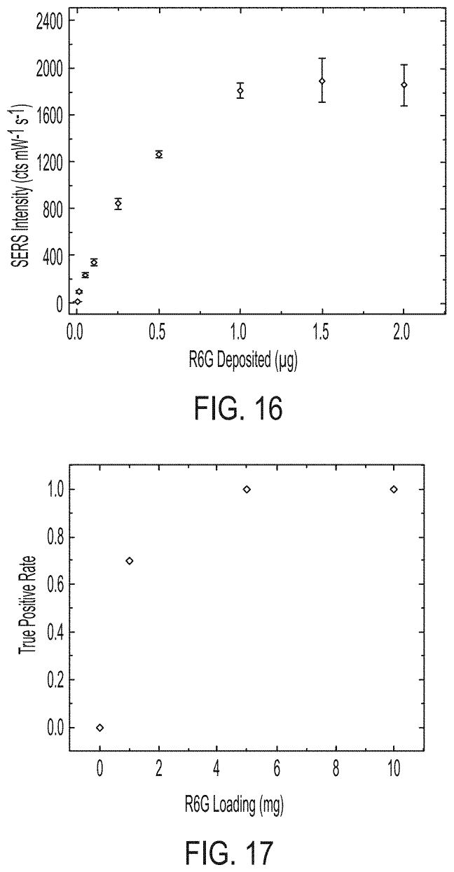

The mean SERS intensity of samples containing R6G was similar at all R6G loadings tested. This could be because even the lowest loading saturated the SERS-active surfaces of the nanopaper. In this case, additional R6G would benefit less from SERS and have little effect on the overall signal. To test this, aqueous R6G solutions were deposited on nanopaper samples and the SERS response was measured (FIG. 16). The results showed that as more R6G was deposited on the nanopaper, the corresponding increase in SERS intensity diminished; beyond 1 μg, additional R6G did not increase the SERS intensity, indicating saturation of SERS-active surfaces. This maximum intensity value agreed with the intensities observed in VF-PC-SERS experiments. The saturation level of 1 μg means that even at the lowest loading tested, VF-PC-SERS experiments could present regions with surface saturation if the filtration captures as low as 0.1% of the total R6G load.

The consistency in SERS intensity implies that the dynamic range for this system is narrow (between 0 and 0.6 mg m⁻³ R6G) and lies outside the conditions tested. This means that the protocol is not suitable for quantitative detection. This is not a major setback for many potential applications of VF-PC-SERS; for rapid identification of illicit or hazardous substances in the field, qualitative detection is sufficient. Positive qualitative detection is achieved when the mean intensity of a VF-PC-SERS experiment exceeds a detection threshold, which was defined as 3 standard deviations above the mean of the negative controls. FIG. 15 shows that with 60 s VF duration, VF-PC-SERS achieved 100% sensitivity at R6G loadings. It is also useful to define a qualitative limit of detection as the lowest analyte loading which reliably results in positive detection. If reliable detection is at 100% sensitivity, then the qualitative limit of detection for this experiment is ≤0.6 mg m⁻³.

To further test the limits of qualitative detection, the VF duration was reduced to 20 s and repeated the VF-PC-SERS protocol for R6G loadings of 10, 5, 1, and 0 mg (equivalently 6, 3, 0.6, and 0 mg m³). With reduced vacuum time, fewer particles were collected on the nanopaper surface, reducing the R6G signal. Loadings of 6 and 3 mg m⁻³ maintained 100% sensitivity, but at 0.6 mg m⁻³, detection became unreliable (FIG. 17). Therefore, for 20 s filtration duration, the qualitative limit of detection was between 0.6-3 mg m³.

Detection in Complex Matrices.

VF-PC-SERS provides a means for sample collection and applications outside the laboratory, which often involve unclean, diluted, or otherwise adulterated substances. Therefore, the method is able to collect potentially contaminated samples and identify the substance of interest. To simulate the collection and identification of adulterated substances, R6G powder was mixed with two diluents: topsoil and D-lactose powder. Topsoil was chosen to demonstrate specificity in generally unclean environments, such as outdoors, and lactose was selected as a common diluent for illegally distributed controlled substances. In specificity experiments, filtration duration was 60 s and R6G loading remained constant at 5 mg while the diluent loading varied. Lactose and topsoil were tested at 5, 7.5, 11.7, 20, 45, 95, and 1000 mg loadings, corresponding to powder compositions of 50, 40, 30, 20, 10, 5, and 0.5% R6G (w/w), respectively. Topsoil was also tested at 5 g, or a powder composition of 0.1% R6G (w/w). Three experimental replicates were performed for each loading of both diluents, after which a negative control was performed wherein the chamber was loaded with the same level of diluent containing no R6G.

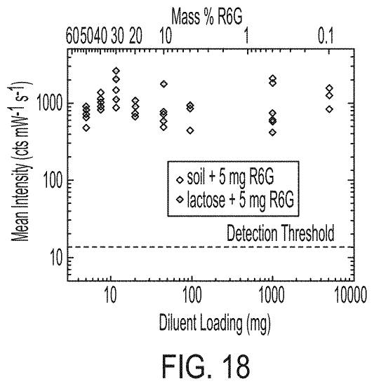

Vacuum filtration of contaminated samples resulted in accumulation of R6G and the diluent powder on the nanopaper surface. For diluent loadings ≥95 mg, the accumulation could be seen by eye. After elution with methanol, in which lactose is only sparingly soluble, a distinct pink colored band separated from the collection area, migrating with the solvent front. This visual indicator of R6G was not observed in any negative control samples. SERS analysis at the solvent front showed strong intensity at 612 cm⁻¹ in all non-control samples and showed negligible intensity at 612 cm⁻¹ in all control samples (FIG. 18).

The SERS results of FIG. 18 demonstrate that VF-PC-SERS is a robust platform for the detection of R6G despite severe contamination by multiple diluents. No decrease in SERS intensity was observed as diluent loading was increased. Because all contaminant loadings resulted in correct identification of R6G, the specificity limit was not fully determined; it was shown that R6G identification is specific for powder mixtures ≥0.5% in lactose and ≥0.1% in topsoil. This result compares favorably with the thermal desorption direct analysis in real time mass spectrometry (TD-DART-MS), which could detect solid fentanyl dispersed in dirt simulant at 0.1 wt. %.

Example of Detected Rhodamine 6G (R6G) Chemical Dust from Air Using Vacuum.

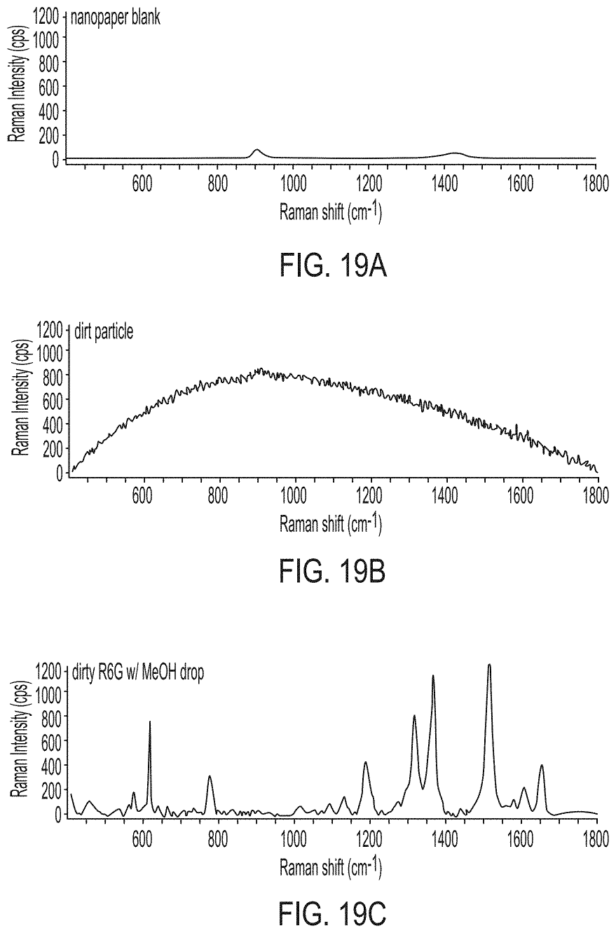

A sample was placed inside a ˜10 L vacuum chamber with and the vacuum line was blocked by a nanopaper filter. A vacuum pump was then switched on for several minutes, with intermittent agitation of the chamber to help raise dust. The R6G dusts were collected by the nanopaper. FIG. 19A illustrates Raman spectrum of the clean nanopaper. FIG. 19B illustrates Raman spectrum of the nanopaper after filtering the garden soil. The garden soil produced a significant background signal. FIG. 19C illustrates the Raman spectrum of the nanopaper after filtering the mixture of R6G and garden soil. After applying a drop of solvent, the background signal could be removed, and the clear R6G Raman spectrum was observed.

Example of Limit of Detection (LoD) for R6G Chemical Dust Detection in Air.

To determine the limit of detection, a larger chamber (˜1.7 m³) was constructed of metal frame and plastic sheeting (FIG. 12). In this experiment, a carefully measured amount of R6G powder was placed in the chamber, and a vacuum pump outfitted with a nanopaper filter was inserted and switched on. The dust sample was then dispersed within the chamber by a jet of compressed air; four fans in the corners of the chamber helped to circulate the dust cloud. Collection continued for 10 minutes, after which the nanopaper was removed and analyzed by Raman spectroscopy.

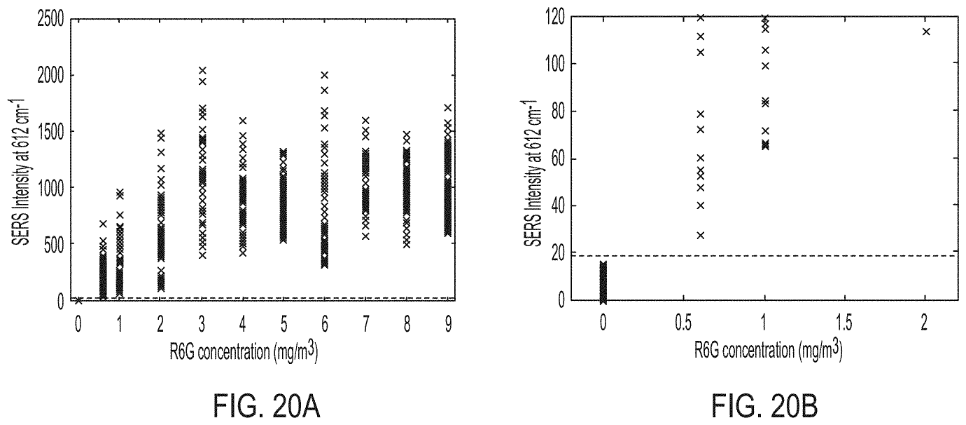

The common criterion for limit of detection is the lowest concentration which produces a signal greater than the detection threshold μ_{b}+3α_{b}, where μ_{b} and σ_{b} are the mean and standard deviation of a blank sample. To determine the lower threshold of reliable detection the experiment was performed without R6G dust as well as with different amounts of R6G dust (FIG. 20). All measurements for the lowest non-blank point (0.6 mg/m³) exceeded this threshold, so the LoD for R6G in this system was <0.6 mg/m³.

As shown above, it has been demonstrated that nanopaper can be used as a tri-functional substrate to capture solids dispersed in air, separate the captured materials by chromatography, and enhance their Raman scattering signal to allow SERS identification. The combination of these functions makes VF-PC-SERS a simple and rapid method, with each function being compatible with on-site analysis of solid specimens. Although only one target species, R6G, was tested in the presented VF-PC-SERS experiments, the promising sensitivity and specificity observed indicates good potential for use in more applied scenarios, including narcotics testing. In view of the above, hazard chemicals (such as fentanyl and its analogs) could be tested and identified, the system could be used remotely, via, for example robotic vacuum cleaners and handheld Raman spectrometers, and single-use VF-PC-SERS cartridges incorporating a nanopaper test strip and elution solvent could be utilized.

### Additional Applications

Nanopaper Modified by 1-Butanetiol Enhanced the Signal of Fentanyl.

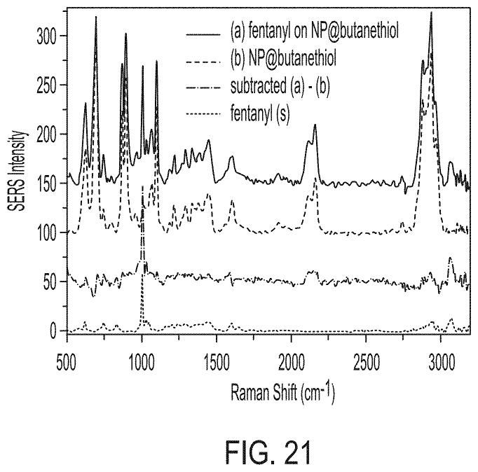

FIG. 21 illustrates the SERS results for trace fentanyl detection. Spectra are shown for fentanyl adsorbed on nanopaper modified with 1-butanethiol, a control nanopaper modified with 1-butanethiol, and the difference spectrum. Solid fentanyl spectrum is given for comparison. Spectra have been offset for visual clarity. As shown in FIG. 21, fentanyl detection is enhanced with the addition of 1-butanethiol, as the SERS intensity is higher for fentanyl on nanopaper with 1-butanethiol. This shows promising results for the use of SERS for fentanyl detection, even when only trace amounts are present. In some embodiments, the nanopaper can be modified with a compound to enhance affinity between the substrate and the analyte (e.g. fentanyl) to thereby increase detection limit. In some embodiments, the compound can be organic compounds, inorganic compounds, thiols, functional groups, functional molecules, 1-butanethiol, or combinations thereof.

Disposable Threat Detection Cartridge.

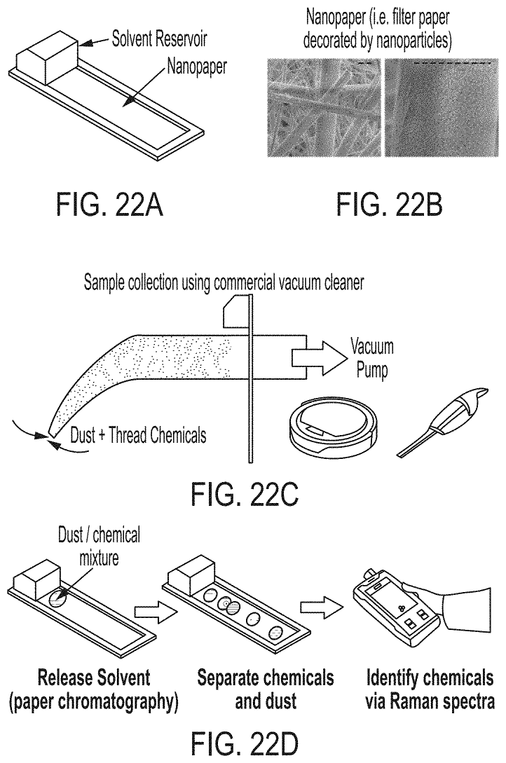

FIG. 22 illustrates an example disposable threat detection cartridge. FIG. 22A illustrates that a plastic cartridge contains an organic solvent reservoir and nanopaper. The nanopaper is a glass fiber paper decorated with silver nanoparticles. This nanopaper has multiple functions. First, it serves as a vacuum filter paper for sample collection. Second, it is the stationary phase for paper chromatography. Third, the nanoparticles on the nanopaper enhance Raman signals of chemicals, improving detection sensitivity. FIG. 22B illustrates an SEM image of nanopaper (scale bar=4 μm). FIG. 22C illustrates that the cartridge can be installed in commercial vacuum cleaners, including handheld or robotic vacuum cleaners. Dusts and chemicals (e.g., drug, explosive, etc.) on the floor or in the air will be collected on the nanopaper. FIG. 22D illustrates separating dust and chemicals to improve the detection limit. The solvent reservoir is punctured to release organic solvents (e.g., methanol or ethanol). The components move with the flowing solvent, leading to separations of components. Inserting the cartridge in to a handheld Raman spectrometer can then identify the chemicals via their fingerprint spectra. In some embodiments, the disposable threat detection cartridge can be used to identify fentanyl, or fentanyl-related compounds (e.g., carfentanil and acetyl fentanyl), natural opioids, or synthetic derivatives thereof.

Trace Detection of Opioid Residues on Mail Parcels.

Various aspects of the nanoplasmonic paper substrates of the present disclosure allow for the collection and detection of a trace amount of opioid residues left on contaminated mail packages.

To non-intrusively inspect illicit mail parcels, opioid residues left on contaminated mail parcels can be detected. In some embodiments, a multi-functional instrument collects and detects opioid residues. This instrument can include three components, including, but not limited to: (1) a commercial Raman analyzer for opioid identification; (2) a commercial vacuum filtration system to collect opioid residues; and (3) a special detection cartridge to improve detection accuracy of Raman spectroscopy. To inspect a pile of 100 parcels, the entire detection process takes less than 1 minute, leading to 0.6 sec of detection time per parcel. This technology can be easily modified and then be adapted by law enforcement or border patrol agents to inspect other items, such as, but not limited to, luggage and trucks crossing borders. This will assist not only the postal service and/or package handing services in mail parcel inspection, but also other federal government agencies in border security and drug trafficking control.

The systems and methods of the present disclosure can utilize an assumption that illicit drug dealers will contaminate mail parcels when they pack opioids in boxes. This assumption is the same as the hypothesis of trace explosive detectors used in airport security, which detect trace explosives left on contaminated hands or baggage. Based on this assumption, disclosed herein are systems and methods that can non-intrusively collect and detect opioid residues left on surfaces of mail parcels.

In some embodiments, the system includes components, such as, but not limited to: (1) a Raman analyzer for opioid identification; (2) a vacuum filtration system (e.g. vacuum cleaner) to collect opioid residues left on contaminated parcels; and (3) a multi-functional detection cartridge to dramatically improve detection accuracy of Raman spectroscopy. Raman spectroscopy is selected because it can identify different substances via molecular fingerprint spectra, including common drugs of abuse, cannabinoids, fentanyl, and other new psychoactive substances (e.g. TRUNARC™ can identify over 240 types of drugs). However, the detection accuracy of Raman analyzer is very poor when opioids are mixed with complex background matrices. When opioid residues are collected from mail parcels, dirt, drug adulterants, excipients, and other packaging materials can hinder opioid signals, leading to false negative results. To overcome this intrinsic barrier, a disposable detection cartridge that contains a piece of “nanopaper”, a glass microfiber filter coated with a dense layer of silver nanoparticles, as previously discussed, was developed. This nanopaper provides multiple functions. First, silver nanoparticles offer Surface Enhanced Raman Spectroscopy (SERS), leading to 10⁵-fold enhancement of Raman signals over typical Raman detectors. This enhancement allows for the detection of trace amounts of opioids (e.g. able to detect a single airborne particle of fentanyl powder). Second, nanopaper serves as a stationary phase for paper chromatography. Via chromatography separation, low concentration opioids can be separated from abundant ingredients, minimizing interference from background matrices. Third, nanopaper can serve as a vacuum filter paper, resulting in concentrated sample collection via vacuum filtration.

Operations (e.g. sample collection, separation, and detection) can be conducted in a single detection cartridge; thus, an automated operating system to conduct parcel inspection can be created. The automated operation not only reduces human error, but also allows remote control of the instrument. It is worth noting that the remote operation can reduce the risk of exposure to highly potent opioids (e.g. fentanyl), maintaining occupational safety.

The systems and methods, as disclosed herein, are highly flexible. A Raman detector and a detection cartridge can be integrated with a high-pressure air jet machine to screen a pile of mail parcels. It takes approximately 1 minute to sweep 100 parcels. Thus, the systems and methods disclosed herein can achieve 0.6 seconds detection time per parcel. In addition, air jet machines can be installed on mail sorting machinery to screen mail parcels one-by-one. Moreover, handheld detectors to assist law enforcement and border patrol agents in field-testing can also be developed, as discussed above. The high flexibility will allow federal agencies to redeploy the technology.

Commercial Raman analyzers can identify over 240 different types of drugs. However, commercial Raman analyzers cannot detect a trace amount of drugs mixed with complex background matrices. Detection cartridges as disclosed herein overcome these barriers and improve the detection sensitivity and accuracy by at least 4 orders of magnitude. The detection cartridges herein can, for example: (1) collect and detect airborne fentanyl powders at very low concentration, such as, but not limited to, approximately <0.6 ppb; (2) have about 100% detection accuracy, for example, 0.1 wt % of airborne fentanyl mixed with 99.9% of topsoil (dirt) or lactose (drug adulterant) was detected with 100% accuracy; (3) maintain good limit of detection, for example, the instruments presented herein can detect a single 20 μm-sized particle of fentanyl powder, and as such, as long as more than one drug particle from a mail package surface can be collected, the systems and methods are sensitive enough to identify it; (4) nanopaper can be fabricated in large quantities; and (5) the detection cartridges can be used in any commercial vacuum pumps/cleaners and Raman analyzers, such that, the cartridges as disclosed herein can be immediately available for different agencies.

In some embodiments, detection time can be less than 0.5 seconds per parcel. In some embodiments, the limit of detection can be less than 1 g per parcel. In some embodiments, the detection systems and methods presented herein can provide less than 1% false negatives. In some embodiments, the detection systems and methods presented herein can provide less than 1% false positives.

The detection systems and methods of the present disclosures detect opioid residues left on mail parcel surfaces, so the detection systems are not limited by the forms and purity of illicit drugs. Even if, for example, opioids are mixed with abundant drug adulterants or excipients, the detection systems can accurately identify opioids. Based on the discussions and demonstrations described in detail above the systems and methods presented herein can detect less than 0.1 wt % of drug with 100% accuracy. Because these systems and methods detect opioid residues left on mail parcel surfaces, the packaging materials will not affect the detection capability.

In some embodiments, a high-pressure air jet can be utilized to sweep a pile of parcels. A single inspection can take less than 1 minute. If a pile contains 100 parcels, the detection time will be less than 0.6 seconds per parcel. In some embodiments, the detection systems can include an automated operating system, allowing operators to control the inspection process remotely. Once the detection system identifies a suspicious parcel, a warning message can be delivered to operators and/or officers, and subsequently, the operators and/or officers can remove the suspicious parcel from the mail sorting system and perform a closer inspection. In some embodiments, the automated operating system can control all operation procedures, including sample collection, chromatography separation, and Raman spectra identification. Furthermore, in some embodiments, a computer program can automatically compare spectra of unknown substances with the spectral library such that operators do not need to interpret the data.

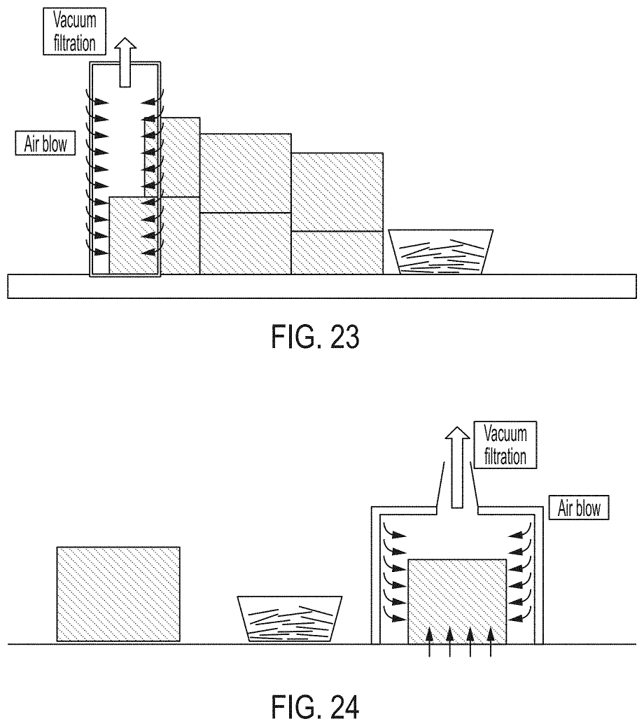

In some embodiments, as shown in FIG. 23, a high-pressure air jet machine is utilized to screen piles of unloaded mail parcels. In this particular embodiment, the detection system can detect a large pile of mail parcels automatically, and can, for example, scan approximately 100 packages in about 1 minute. In some embodiments, as shown in FIG. 24, the air jet machine can be installed in a sorting railroad to inspect parcels one-by-one. Moreover, a handheld detector, as previously described, can be used to inspect parcels manually. The handheld system can be used for closer inspection when a suspicious mail parcel is identified. In some embodiments, the detection systems can be operated manually. In some embodiments, the detection systems can be operated automatically. In some embodiments, the detection systems can include an automated operating system for routine inspection. In various embodiments, a large-size air jet machine with the detection equipment to screen a large pile of parcels can be integrated into the detection systems described herein. In addition, the detection systems described herein can utilize a comprehensive spectral library in order to detect different types of opioids, cannabinoids, new psychoactive substances, and other controlled substances.

In an embodiment, the present disclosure pertains to a method to detect trace amounts of fentanyl. In some embodiments, the method includes contacting a sample on a substrate, where the substrate is a nanoplasmonic paper, performing surface-enhance Raman scattering detection with paper chromatography separation of the substrate, and responsive to the performing, identifying fentanyl in the sample. In some embodiments, the nanoplasmonic paper includes glass microfiber filter paper that has been coated with a dense layer of silver nanoparticles. In some embodiments, the dense layer of silver nanoparticles is coated on the nanoplasmonic paper via a silver mirror reaction. In some embodiments, the substrate includes 1-butanethiol or other functional groups, compounds, molecules, or the like to enhance affinity between SERS substrate and an analyte of the sample thereby increasing detection limit.

In another embodiment, the present disclosure pertains to a method to detect trace amounts of fentanyl. In some embodiments, the method includes contacting a sample on a substrate, where the substrate is a nanoplasmonic paper that can include additional functional molecules, such as 1-butanethiol, performing surface-enhanced Raman scattering detection with paper chromatography separation of the substrate, and responsive to the performing, identifying fentanyl in the sample. In some embodiments, the nanoplasmonic paper includes glass microfiber filter paper that has been coated with a dense layer of silver nanoparticles.

In a further embodiment, the present disclosure pertains to a method to identify mixed-analyte samples. In some embodiments, the method includes contacting a sample on a substrate, where the substrate is a nanoplasmonic paper, performing surface-enhanced Raman scattering detection with paper chromatography separation on the substrate, and identifying mixed-analytes in the sample. In some embodiments, the nanoplasmonic paper includes glass microfiber filter paper that has been coated with a dense layer of silver nanoparticles. In some embodiments, the dense layer of silver nanoparticles is coated on the nanoplasmonic paper via a silver mirror reaction.

In another embodiment, the present disclosure pertains to a method including using a silver-coated glass microfiber filter paper as a combination paper chromatographer/surface-enhanced Raman scattering substrate. In a further embodiment, the present disclosure pertains to a method to collect suspicious substances spread in the air or on the floor, separate analytes of interest from complex background ingredients or dirt, and improve the limit of Raman detection on a single substrate, the method including using a nanoplasmonic paper as a substrate. In some embodiments, the using includes operating a vacuum with the nanoplasmonic paper as a filter.

In an embodiment, the present disclosure pertains to an apparatus to collect suspicious substances. In some embodiments, the apparatus includes a vacuum pump coupled to a filtration unit, and the filtration unit includes a filter substrate, where the filter substrate includes a nanoplasmonic paper. In some embodiments, the apparatus provides for a method of collecting suspicious substances that minimizes risks of exposure to fentanyl or other threat chemicals potentially exposed to law enforcement. In some embodiments, the apparatus provides for a method of collecting suspicious substances that reduces human error during collection and transfer of the suspicious substances.

In an additional embodiment, the present disclosure pertains to a filter cartridge including a filter substrate including nanoplasmonic paper where the filter cartridge is adaptable to be coupled to a vacuum. In another embodiment, the present disclosure pertains to a disposable threat detection cartridge including a first end and a second end, a solvent reservoir coupled to the first end, and a nanopaper filter extending from the solvent reservoir to the second end, where the disposable threat detection cartridge is operable to be installed as a filter in a commercial vacuum, a handheld vacuum, a robotic vacuum, or the like. In some embodiments, the nanopaper filter is a glass fiber paper decorated with silver nanoparticles. In some embodiments, the nanopaper filter is nanoplasmonic paper. In some embodiments, the solvent reservoir can be punctured to release a solvent. In some embodiments, the disposable threat detection cartridge is capable to be inserted into a handheld Raman spectrometer.

Although various embodiments of the present disclosure have been illustrated in the accompanying Drawings and described in the foregoing Detailed Description, it will be understood that the present disclosure is not limited to the embodiments disclosed herein, but is capable of numerous rearrangements, modifications, and substitutions without departing from the spirit of the disclosure as set forth herein.

The term “substantially” is defined as largely but not necessarily wholly what is specified, as understood by a person of ordinary skill in the art. In any disclosed embodiment, the terms “substantially,” “approximately”, “generally”, and “about” may be substituted with “within [a percentage] of” what is specified, where the percentage includes 0.1, 1, 5, and 10 percent.

The foregoing outlines features of several embodiments so that those skilled in the art may better understand the aspects of the disclosure. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure, and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a”, “an”, and other singular terms are intended to include the plural forms thereof unless specifically excluded.

What is claimed is:

1. A method to detect analytes, the method comprising:
contacting a sample on a substrate, wherein the substrate is a nanoplasmonic paper;
performing surface-enhanced Raman scattering detection with paper chromatography separation on the substrate; and
identifying at least one analyte in the sample.

2. The method of claim 1, wherein the nanoplasmonic paper comprises glass microfiber filter paper coated with silver nanoparticles.

3. The method of claim 2, wherein the silver nanoparticles are coated on the nanoplasmonic paper via a silver mirror reaction.

4. The method of claim 1, wherein the substrate comprises a compound to enhance affinity between the substrate and the at least one analyte to thereby increase detection limit.

5. The method of claim 4, wherein the compound is selected from the group consisting of organic compounds, inorganic compounds, thiols, functional groups, functional molecules, 1-butanethiol, or combinations thereof.

6. The method of claim 1, wherein the sample is a mixed-analyte sample comprising a plurality of analytes.

7. The method of claim 1, wherein the contacting comprises collecting solid particles of the sample onto the substrate via vacuum.

8. The method of claim 7, wherein the nanoplasmonic paper is extended between a first end and a second end of a filter cartridge adaptable to be coupled to a vacuum; and
wherein a solvent reservoir is coupled to the first end.

9. The method of claim 1, wherein the performing comprises:
eluting a solvent through the nanoplasmonic paper;
separating components of the sample; and
identifying the components of the sample via spectroscopy.

10. The method of claim 1, wherein the at least one analyte is selected from the group consisting of natural opioids, synthetic opioids, opioid residues, fentanyl, fentanyl-related compounds, carfentanil, acetyl fentanyl, cannabinoids, synthetic cannabinoids, or combinations thereof.

11. An apparatus to detect analytes, the apparatus comprising:
a vacuum pump coupled to a filtration unit operable to collect solid particles off an object; and
the filtration unit comprising a filter substrate, wherein the filter substrate comprises nanoplasmonic paper.

12. The apparatus of claim 11, wherein the filter substrate is disposed with a filter cartridge, the filter cartridge comprising:
a first end and a second end;
a solvent reservoir coupled to the first end; and
the nanoplasmonic paper extending on a surface of the filter cartridge from the solvent reservoir to the second end.

13. The apparatus of claim 12, wherein the solvent reservoir is operable to be punctured to elute a solvent through the nanoplasmonic paper thereby separating components of the solid particles.

14. The apparatus of claim 12, wherein the filter cartridge is operable to be inserted into a handheld Raman spectrometer.

15. The apparatus of claim 11, comprising a sorting surface and at least one air jet in fluid communication with the object and the filtration unit.

16. The apparatus of claim 11, wherein the object is selected from the group consisting of a surface, luggage, a mail parcel, carpet, wood, cloth, or combinations thereof.

17. The apparatus of claim 11, wherein the filter substrate comprises a compound to enhance affinity between the filter substrate and analytes in the solid particles to thereby increase detection limit of the analytes.

18. The apparatus of claim 17, wherein the compound is selected from the group consisting of organic compounds, inorganic compounds, thiols, functional groups, functional molecules, 1-butanethiol, or combinations thereof.

19. The apparatus of claim 11, wherein the nanoplasmonic paper comprises glass microfiber filter paper coated with silver nanoparticles.

20. The apparatus of claim 19, wherein the silver nanoparticles are coated on the nanoplasmonic paper via a silver mirror reaction.
